(19) United States
(12) Patent Application Publication
(10) Pub. No.: US 2005/0113744 A1
(43) Pub. Date: May 26, 2005

(54) Agent delivery systems and related methods under control of biological electrical signals

(75) Inventors: John P. Donoghue, Providence, RI (US); J. Christopher Flaherty, Topsfield, MA (US); Gerhard M. Friehs, East Greenwich, RI (US); Brian W. Hatt, Salt Lake City, UT (US); Mijail D. Serruya, Providence, RI (US); Maryam Saleh, Providence, RI (US)

Correspondence Address: Leslie I. Bookoff;FINNEGAN, HENDERSON, FARABOW,, GARRETT & DUNNER, L.L.P., 1300 I. Street, N.W., Washington, DC, 20005-3315, US

(73) Assignee: Cyberkinetics, Inc.

(21) Appl. No.: 10/717,924

(22) Filed: Nov. 21, 2003

(51) Int. Cl. A61M 31/00

(52) U.S. Cl. 604/66

(57) ABSTRACT

Systems and methods are disclosed for detecting neural, biological, or other electrical signals generated within a patient's body and processing those signals to generate a control signal that may control the delivery of a biologic, therapeutic, or other agent, such as a drug. Embodiments include a system having a sensor implanted in a patient's brain to detect neural signals used to control delivery of a drug to the patient. The system may also control an internal and/or external device, such as a prosthetic limb, and control delivery of a drug to increase the performance of the system and/or the controlled device.

### FIELD OF THE INVENTION

The present invention relates to systems and methods for delivering drugs or other agents, and, more particularly, to systems and methods for delivering agents based on biological electrical signals.

### BACKGROUND OF THE INVENTION

Drug treatment of a patient often requires performing one or more diagnostic tests to determine, among other things, the appropriate type, amount, and/or delivery rate of one or more drugs to the patient. Diagnostic tests also may be used to determine the adverse side effects of a drug therapy or the beneficial effects of that therapy, so as to modify the therapy by type, amount, and/or rate of drug delivery, for example. These diagnostic tests often include body fluid analyses, such as blood or urine tests, or other types of invasive or noninvasive tests, such as x-rays, MRIs, endoscopic or laparoscopic procedures to, for example, obtain tissue biopsies, or other surgical, percutaneous, or endovascular diagnostic procedures that result in a drug therapy.

Moreover, certain types of diseases or conditions may require continual or periodic diagnostic testing to determine the appropriate drug therapy at a given time. These diseases and conditions include, for example, cancer (which may require periodic chemotherapy) and diabetes (which may require careful monitoring of blood glucose level and insulin therapy). There is a continuing need for alternative diagnostic testing or other methods to determine the appropriate drug therapy for a patient at any given time.

Recent advances in neurophysiology have allowed researchers to detect and study the electrical activity of highly localized groups of neurons with high temporal accuracy and in specific locations in the brain. The information in the sensed electrical activity may include a variety of information, including physiologic information and motor control information. These advances have created the possibility of extracting and processing that information and creating brain-computer interfaces that, for example, may allow an amputee to control a prosthetic limb in much the same way that the amputee would control a natural limb.

Various sensors have been used to detect electrical activity in a body, and specifically the brain. Noninvasive sensors, such as multichannel electroencephalogram (EEG) sensors placed on the surface of a person's skin, have been used as simple brain-computer interfaces. EEG sensors may not offer sufficient temporal or spatial resolution needed, for example, for prosthetic control, as such noninvasive sensors detect mass fluctuations of neuron activity that have been attenuated by the intervening bone and tissue. As a result, these types of brain-computer interfaces derive more simple forms of information from the neuron activity. They also operate relatively slowly because the mass neuron signal activity modulates at very low rates, requiring more processing time.

More advanced brain-computer interfaces use sensing electrodes placed directly in contact with the brain to detect neuron activity. These electrodes, which may comprise a micro-wire or hatpin-like electrode, each form a recording channel that may directly detect the electrical impulse signal from all of the neurons in the electrode's vicinity. Further signal processing may then isolate the individual neuron signals, each of which comprises a series of electrical spikes reflecting information correlated to a respective function (e.g., a particular movement of a particular limb). The brain encodes this information according to, for instance, the frequency or firing rate of the spikes. By collecting the firing rates of a number of individual neuron signals detected via a number of recording channels, a brain-computer interface can derive control signals to control, for example, a neural prosthetic device.

Many types of therapeutic devices, including brain-computer interfaces, can be implanted into and/or on the body, such as muscle stimulators, magnetic therapy devices, or drug delivery systems. A number of such devices may also be implanted where the different implants may then communicate with one another.

### SUMMARY OF THE INVENTION

According to a first aspect of the invention, a method for treating a body is disclosed that includes detecting electrical signals from a first part of the body, processing the detected electrical signals to generate a first control signal, controlling a device based on the first control signal, generating a second control signal, and providing information relating to delivery of an agent to the body. The information is based on the second control signal.

According to another aspect, a system for treating a body is disclosed. The system includes a sensor configured to be proximate to a first part of the body generating electrical signals and to detect the electrical signals, a first processor connected to the sensor for processing the detected electrical signals to generate a first control signal, a device configured to receive the first control signal and be controlled by the first control signal, and a second processor configured to generate a second control signal based on a monitored parameter of the device and to provide information relating to delivery of an agent to the body based on the second control signal.

According to yet another aspect, a method for treating a body is disclosed that includes detecting neurological signals transmitted to a first part of the body, wherein the detected neurological signals relate to secretion of a first agent within the body. The method further includes processing the detected neurological signals to generate a first delivery control signal, and delivering a second agent to the body based on the first delivery control signal.

According to a still further aspect, a method for treating a body is disclosed that includes detecting electrical signals generated by a first part of the body, processing the sensed electrical signals to generate a performance value reflecting a performance of an applied treatment to the body, determining a delivery control signal based on the performance value, and delivering an agent to the body based on the delivery control signal.

According to another aspect, a method for treating a body is disclosed that includes detecting electrical signals from a part of the body, processing the detected electrical signals to generate a first control signal, controlling a device based on the first control signal, monitoring a parameter of the device, comparing the parameter to a value, generating a second control signal based on the comparison of the parameter to the value, and providing information relating to delivery of an agent to the body. The information is based on the second control signal.

According to yet another aspect, a method for treating a body is disclosed that includes detecting electrical signals from a part of the body, processing the detected electrical signals to generate a first control signal, controlling a device based on the first control signal, monitoring a parameter of at least one of the detected electrical signals and the first control signal, comparing the parameter to a value, generating a second control signal based on the comparison of the parameter to the value, and providing information relating to delivery of an agent to the body. The information is based on the second control signal.

According to still another aspect, a system for treating a body is disclosed that includes a sensor configured to be proximate to a first part of the body generating electrical signals and to detect the electrical signals, a first processor connected to the sensor for processing the detected electrical signals to generate a first control signal, a device configured to receive the first control signal and be controlled by the first control signal, and a second processor configured to generate a second control signal based on a measured parameter of at least one of the detected electrical signals and the first control signal. The second processor is configured to provide information relating to delivery of an agent to the body based on the second control signal.

Both the foregoing general description and the following detailed description are exemplary and are intended to provide further explanation of the embodiments of the invention as claimed.

### BRIEF DESCRIPTION OF THE DRAWINGS

The accompanying drawings, which are incorporated in and constitute a part of this specification, illustrate various embodiments of the present invention, and, together with the description, serve to explain the principles of the invention. In the drawings:

FIG. 1 illustrates an agent delivery system consistent with the present invention;

FIG. 2 illustrates an exemplary embodiment of a brain implant system consistent with the present invention;

FIGS. 3 and 4 illustrate exemplary embodiments of an agent delivery unit consistent with the present invention;

FIGS. 5A-5C show flow diagrams of exemplary methods for delivering an agent based on information generated within a patient's body; and

FIGS. 6A-6C show flow diagrams of exemplary methods for delivering an agent to a patient to improve performance of a neurally controlled device.

### DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

Systems and methods consistent with the invention detect neural, biological, or other electrical signals generated within a patient's body, and process those signals to generate a control signal that may control the delivery of a biologic, therapeutic, or other agent, such as a drug. In one exemplary embodiment, a brain-machine interface may be implanted in a patient's brain to detect neural signals used to control delivery of a drug to the patient. In another exemplary embodiment, the brain-machine interface may be used in conjunction with the control of an internal and/or external device, such as a prosthetic limb. For example, the brain-machine interface may control the prosthetic limb based on the patient's detected neural activity associated with an intended movement. In this arrangement, the brain-machine interface may also control delivery of a drug to increase the performance of, for example, the brain-machine interface or the controlled device. Thus, the control signal to the device may change as the performance of the brain-machine interface may change.

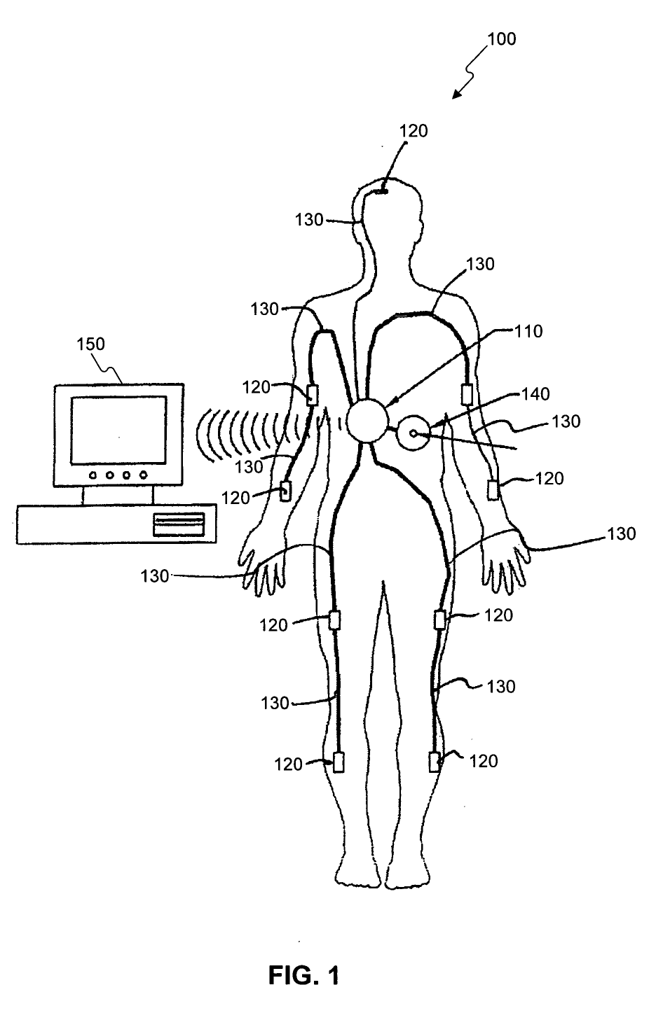

FIG. 1 shows a system 100 of implants for delivering a biologic, therapeutic, or other agent, such as a drug to a patient, according to an exemplary embodiment of the invention. System 100 includes a central implant 110 placed, for example, within the abdomen and connected to various remote implants 120 arranged throughout the body via connectors 130.

Central implant 110 may serve one or more of a variety of functions, including receiving, processing, and/or transmitting electrical signals, processing electrical signals to create one or more control signals, including a drug delivery control signal, transmitting control signals throughout the body, and/or sending or receiving power to or from other implants 120 located throughout the body. Central implant 110 also may include an implanted or external pump (not shown) that may, for example, deliver an agent, such as a drug, to another implant or other part of the body. If an implanted pump is used, a remote signal could be used to control the pump via a wired connection (not shown) or a wireless connection. As described below, the remote control signal (such as a “start” and “stop” signals) could be sent by a brain-machine interface. Implant 110 may also include external drug delivery patches and other like structures that deliver drugs through the skin. The patch could also use programmable transdermal technologies, such that the patch may be programmed to receive a control signal and release drug into the skin based on that signal.

As shown in FIG. 1, central implant 110 may be connected to a reservoir 140 for receiving a refillable supply of drugs. Reservoir 140 may be implanted in the patient or located externally. If implanted, reservoir 140 may be refillable through, for example, a port connection at the patient's skin. For instance, the port may comprise a needle accessible port, such as a rubber septum located under the patient's skin. As a further example, a reservoir may be part of an implanted pump, with central implant 110 sending signals to the pump.

Reservoir 140 may store one or more drugs or other agents for delivery to the patient. Reservoir 140 may be sized to include enough drug to allow a sufficient time between refills. When used in conjunction with a pump, reservoir 140 may be maintained at zero or negative pressure, such that the pump is then used to evacuate reservoir 140.

In other arrangements, reservoir 140 may be pressurized (i.e., positive pressure) and include a drug delivery regulator that controls the amount of drug delivered. For example, a pulsatile, variable rate infusion may be achieved through a fluid metering device. Such a device may include an input valve and an output valve, with an expandable fluid accumulator section between the two valves. With the input valve open and the output valve closed, the accumulator may elastically expand to collect a fixed, precise volume of fluid. The input valve may then be closed. When the output valve then is opened, that fixed volume is expelled from the accumulator through a fluid pathway into the patient. The output valve then may be closed and the cycle can repeat. A fixed volume, preferably a clinically small volume is chosen, and multiple pulses may be given to simulate a near continuous, although controllably variable rate.

An example of a device that achieves a fixed, continuous rate may include a precise restrictor attached to a pressurized reservoir, such as a precision orifice or a long capillary tube. The flow rate can be predicted by Poiseuille's Equation: Volume Rate of Flow=πr⁴ (P₁−P₂)/(8ηL), where (P₁−P₂) is the pressure difference between the ends of the capillary tube, L is the length of the capillary tube, r is the radius of the capillary tube, and η is the coefficient of viscosity of the fluid being infused.

Central implant 110 may also communicate with an external processing unit 150. Processing unit 150 may receive, process, and/or display information associated with the delivery of an agent to the patient. For example, processing unit 150 may display the name of an agent delivered, the amount delivered, and the time of delivery. Processing unit 150 may also be used, as described in more detail below, for providing feedback to the patient when evaluating performance of a neural control system. Further, processing unit 150 may receive a signal from the patient used to instruct a physician or other clinician on whether to deliver an agent to the patient. A system according to such an embodiment may not include a drug delivery unit, as the processing unit supplies information to the clinician about, for example, when to deliver an agent, what type of agent, and/or how much agent to deliver.

Also, as noted above, central implant 110 may receive a remote control signal from, for example, processing unit 150, such as for controlling an implanted pump. Central implant 110 may communicate with processing unit 150 via a wired connection (not shown) or a wireless connection. To transmit wirelessly, implant 110 may include a transceiver that may transmit data using “Bluetooth” technology or according to any other type of wireless communication standard, including, for example, code division multiple access (CDMA), wireless application protocol (WAP), or infrared telemetry.

Remote implants 120 may include a sensor for sensing a biological signal of the body and/or may include a delivery unit for delivering an agent, such as a drug, to the patient. When implant 120 includes a sensor, it may be placed in proximity to electrical signals generated by a patient, such as electrical signals a patient may generate or alter by voluntary control (e.g., neural signals corresponding to an intended bodily movement), or electrical signals generated by other organs or tissue of the body. For example, an implant 120 may detect electrical signals emitted by nerves, ganglia or nuclei (such as the vagus and all other cranial nerves, other peripheral nerves involved in both voluntary and autonomic function such as the splanchnic, or pancreatic nerves and their branches), bones or bone marrow, tumors, lymph nodes, thymus, or other organs, including the heart, glands and/or their ducts (e.g. thyroid, parathyroid, salivary and neurogenital), spleen, liver, kidneys, lungs, gallbladder, intestines, uterus, urogenital organs (e.g. prostate, testes and ovaries). An implant 120 also may detect electrical signals emitted by muscles, including skeletal muscles, smooth muscles, and cardiac muscles, as well as the spinal chord and its roots, fat pads and adipose tissue, and the nerves, roots and ganglias entering fat pads.

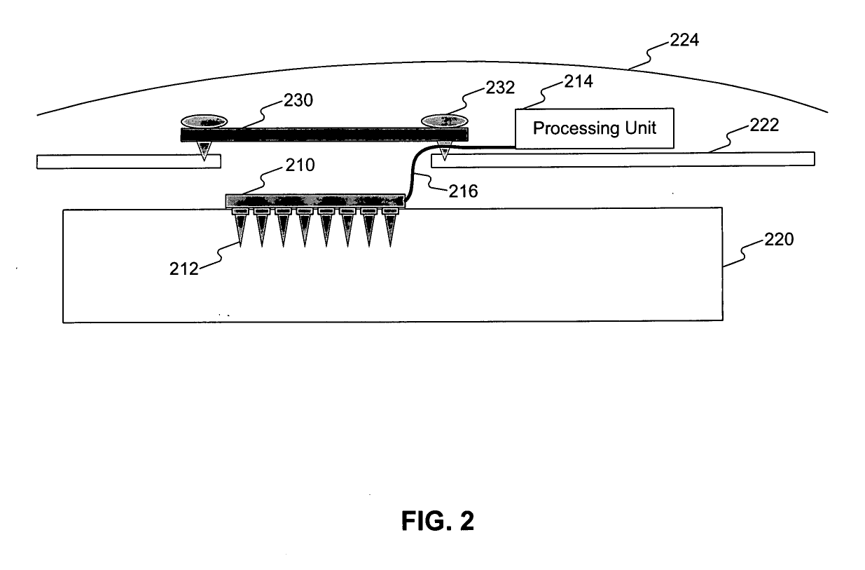

When implant 120 is placed at or near bone marrow, for example, the system may diagnose metabolic acidosis, hypoxia in marrow, or leukemic growth in bone. As a further example, when implant 120 is placed at or near a tumor, it may detect signals relating to tumor cell activity, and when implant 120 is placed at or near, for example the heart or other organ or along any path of the particular organ's neural pathway, systemic effects of chemotherapy may be detected. Further, as shown in FIG. 1, and as described below with respect to FIG. 2, an implant 120 may also be placed directly in contact with the brain to detect neuron activity. In any case, the detected signals may then be used to control delivery of an agent or drug to treat, for example, a condition.

Implant 120 may sense electrical signals using any suitable invasive or noninvasive sensors. For instance, implant 120 may include noninvasive sensors, such as one or more multichannel electroencephalogram (EEG) sensors, placed on the surface of the patient's skin. Implant 120 may also be an invasive sensor, such as that described below with respect to FIG. 2, which may obtain information in the form of neuron spikes, local field potentials (LFPs), or electrocortigram signals (EcoGs). Implants 120 consistent with the present invention, however, may sense or detect other forms of electrical information, or combinations of types of electrical information, depending on, among other things, the type and resolution of the desired information.

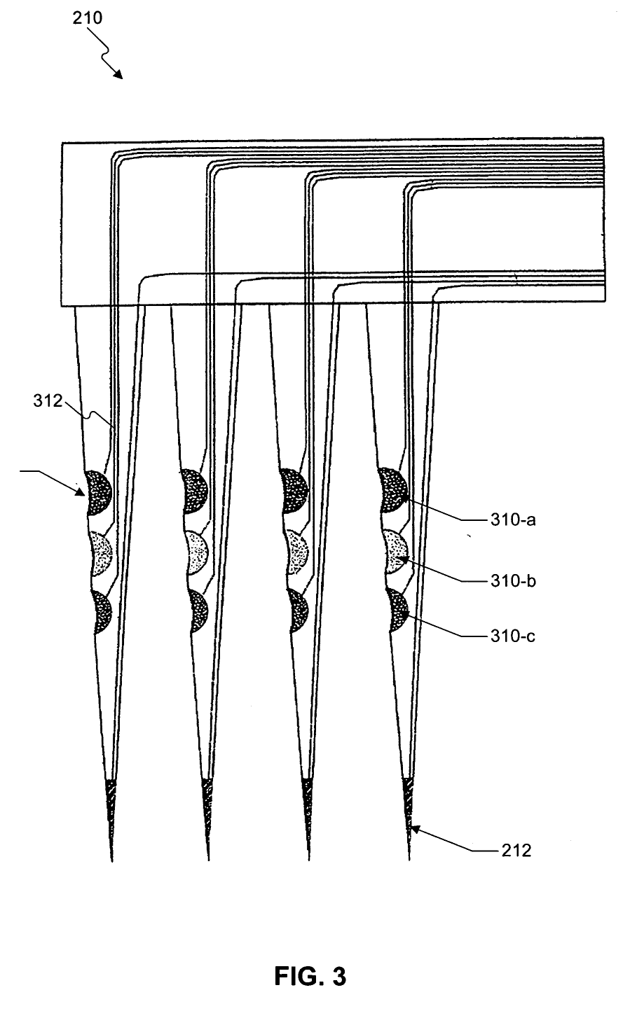

As noted above, implants 120 may also be used to deliver an agent or drug to the patient. As described below in connection to FIGS. 3 to 6, implants 120 may deliver a drug under the control of a delivery control signal generated based on the patient's electrical or biological signals detected by one or more implants 120 within system 100. For example, implant 120 located in the brain may be used to detect neural signals for generating a signal controlling drug delivery. That implant or another implant, such as central implant 110, may then comprise a pump for pumping, based on the control signal, a drug to other implants or locations throughout the body.

Implants 110 and 120 may be connected by connectors 130, which may be optical fibers, metallic wires, telemetry, combinations of such connectors, or some other form of conductors or data transmission. As shown in FIG. 1, connectors 130 may extend into the brain, to the limbs, or through the torso. The implants may also be arranged in a chain configuration.

A system of one or more implants, such as system 100 for example, can be pre-connected prior to implantation or may be connected intra-operatively (e.g., when being implanted within the body during surgery). The optical fibers (and/or cables or electrical conductors) may connect to one or more implants through any suitable method and structure. According to an aspect of the invention, all or substantially all of an implant 110 or 120 may be sealed, i.e. be encapsulated, so that bodily fluids or other foreign matter does not enter the implant. Such a sealed implant may include an optical window for mating with the end of an optical fiber to transmit and/or receive data, information, energy, or the like.

Embodiments of the invention include systems in which various parts may be combined or separated. For example, an implant 120 that senses electrical signals can be combined with the signal processing that generates a control signal. The drug delivery unit also may be combined with the sensor and/or all or part of a signal processing device. For example, devices for amplifying and/or filtering the sensed signals may be combined with the sensor and further processing devices to generate a control signal may be combined with the drug delivery means.

In addition, suitable devices for supplying power to the various parts of system may be combined with one or more parts of that system or connected to parts through, for example, wires or other connecting structure. The system may include one or more power supplies, such as batteries. As known in the art, the power supply may be recharged (e.g., via inductive coupling) or may include power supply systems (such as a typical battery source) that need to be replaced when their power is exhausted.

FIG. 2 generally illustrates a brain implant system consistent with an embodiment of the present invention. As shown in FIG. 2, the system includes an electrode array 210 inserted into a patient's cerebral cortex 220 through an opening in the skull 222. Array 210 may include a plurality of electrodes 212 for detecting electrical brain signals or impulses. While FIG. 2 shows array 210 inserted into cerebral cortex 220, array 210 may be placed in any location of a patient's brain allowing for array 210 to detect electrical brain signals or impulses.

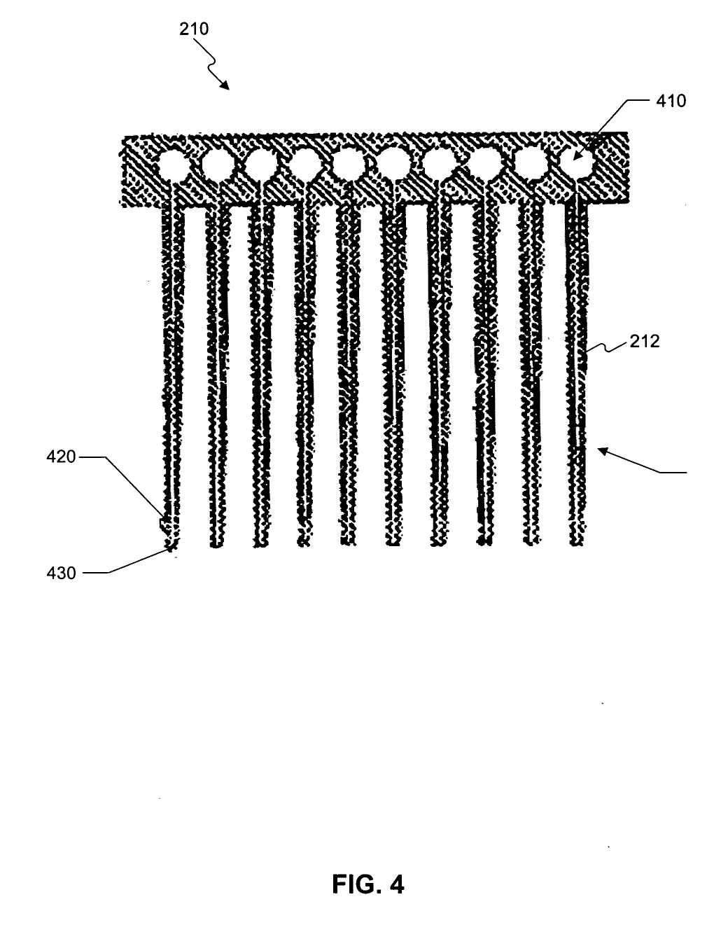

Electrode array 210 serves as the sensor for the brain implant system. While FIG. 2 shows electrode array 210 as eight electrodes 212, array 210 may include one or more electrodes having a variety of sizes, lengths, shapes, forms, and arrangements. Moreover, array 210 may be a linear array (e.g., a row of electrodes) or a two-dimensional array (e.g., a matrix of rows and columns of electrodes). Each electrode 212 extends into brain 220 to detect one or more electrical neural signals generated from the neurons located in proximity to the electrode's placement within the brain. Neurons may generate such signals when, for example, the brain instructs a particular limb to move in a particular way. Electrode array 210 is described in more detail with respect to FIGS. 3 and 4.

U.S. Pat. No. 6,171,239 to Humphrey and entitled “Systems, Methods, and Devices for Controlling External Devices By Signals Derived Directly From the Nervous System” and U.S. Pat. No. 5,215,088 to Normann et al. and entitled “Three-Dimensional Electrode Device” each disclose other arrays suitable for use in connection with systems according to embodiments of this invention. The entire disclosures of those patents are incorporated by reference herein. Other arrays of probes capable of detecting electrical neural signals generated from the neurons may be used with systems according to embodiments of the invention.

In the embodiment shown in FIG. 2, each electrode 212 may be connected to a processing unit 214 via wiring 216. Processing unit 214 may be secured to skull 222 by, for example, the use of an adhesive or screws, and may even be placed inside the skull if desired. A protective plate 230 may then be secured to skull 222 underneath the surface of the patient's skin 224. In exemplary embodiments, plate 230 may be made of titanium and screwed to skull 222 using screws 232, or may comprise a section of skull 222 previously removed and attached to skull 222 using bridging straps and screws (both not shown). However, the invention may use any of a number of known protective plates, such as a biological material, and methods for attaching the same to a patient's skull. Further, processing unit 214 and other surgically implanted components may be placed within a hermetically sealed housing to protect the components from biological materials. Alternative embodiments also include processing unit 214 being included as part of central implant 110 or located external to the patient's body (e.g., as part of processing unit 150 of FIG. 1).

Electrodes 212 transfer the detected neural signals to processing unit 214 over wiring 216. As shown in FIG. 2, wiring 216 may pass out of the opening in skull 222 beneath protective plate 230. Wiring 216 may then run underneath the patient's skin 224 to connect to processing unit 214. Persons skilled in the art, however, will appreciate that arrangements other than the one shown in FIG. 2 may be used to connect array 210 to processing unit 214 via wiring 216.

Processing unit 214 may preprocess the received neural signals (e.g., impedance matching, noise filtering, or amplifying), digitize them, and further process the neural signals to extract neural information that it may then transmit to an external device (not shown), such as a further processing device and/or an agent delivery device. For example, the external device may decode the received neural information into control signals for controlling an agent delivery device or analyze the neural information for a variety of other purposes.

In one exemplary embodiment, processing unit 214 may control the delivery of an agent or drug to the patient. In this respect, processing unit 214 may include an embedded table of values, such as a look-up table of trigger points and/or a look-up table of transfer function variables or coefficients. The stored table values may then be used to convert a detected biological electrical signal to an output control signal. The stored table values may be dependent upon or customized for the patient, a controlled device, or the particular application in which the system is used. The stored table values may also be time dependent or may change as a function of time.

In one embodiment, a clinician, operator, or patient can change the values in the tables. A security control, such as, for example, an electronic password, mechanical key, fingerprint, or retina scan, may be used to gain access to the processing device.

As an example, a particular variable, such as systemic toxicity, may have minimum, maximum and target level values stored in a processor, as well as values (such as a minimum and a maximum) relating to a rate of change of systemic toxicity. A rapid change over a period of time in systemic toxicity (or, for example, other parameters like neural firing rate, neural firing patterns, neural modulation, or organ secretion drive signal), even if the variable stays within maximum and minimum limits, may be an adverse event that the system needs to react to, e.g., trigger an event such as drug delivery.

Processing unit 214 may also conduct adaptive processing of the received neural signals by changing one or more parameters of the system to achieve or improve performance. Examples of adaptive processing include, but are not limited to, changing a parameter during a system calibration, changing a method of encoding neural information, changing the type, subset, or amount of neural information that is processed, or changing a method of decoding neural information. Changing an encoding method may include changing neuron spike sorting methodology, calculations, thresholds, or pattern recognition. Changing a decoding methodology may include changing variables, coefficients, algorithms, and/or filter selections. Other examples of adaptive processing may include changing over time the type or combination of types of signals processed, such as EEG, LFP, neural spikes, or other signal types.

U.S. Pat. No. 6,171,239 to Humphrey and entitled “Systems, Methods, and Devices for Controlling External Devices By Signals Derived Directly From the Nervous System” discloses adaptive processing methodology suitable for use in connection with systems and methods according to embodiments of this invention. As noted above, the entire disclosure of that patent is incorporated by reference herein.

FIG. 3 illustrates an exemplary embodiment of electrode array 210 having reservoirs for delivering agents or drugs. As shown in FIG. 3, electrodes 212 of array 210 may each include one or more reservoirs 310 containing a quantity of a drug for delivery to the patient. Each reservoir 310 may receive a delivery control signal from a control unit (e.g., processing unit 214) via a respective conductor 312. The delivery control signal may then cause a predetermined amount of the drug contained in a reservoir 310 to be delivered to the patient by, for example, causing an opening of reservoir 310 to open, permitting drug to permeate through a membrane at a controlled rate, or otherwise causing drug to be controllably released from reservoir 310.

In operation, a delivery control signal is sent to one or more reservoirs 310 via a respective conductor 312. The delivery control signal may, for example, be a predetermined voltage pulse for a set length of time. The voltage level of the pulse and/or the pulse length may dictate the amount of drug delivered from a reservoir 310. For instance, in one exemplary embodiment, each reservoir 310 includes an outer membrane (not shown). The membrane becomes permeable (i.e., it opens) when a voltage is applied to it via conductor 312. The rate at which the drug passes through the membrane may be based on the value of the voltage level. Thus, the control unit outputting the delivery control signal over conductor 312 can control the amount of drug released based on the control signal's voltage level and/or pulse length. The control signal may also be a continuous signal such that it causes a predetermined flow rate of the drug to be delivered continuously to the patient.

As an example, the permeability of a living cell membrane is affected by electric fields. Drug delivery using living cells loaded with agents may be controlled by electric fields. As a further example, synthetic membranes that are permeable can be used in conjunction with iontophoresis techniques, involving a means of enhancing the flux of ionic compounds across a membrane by the application of an electric current across it.

Reservoirs 310 may also include other types of membranes. For example, reservoirs 310 may store drug at a particular pressure, which in turn causes the drug to permeate through the membrane. To this end, reservoir 310 may be filled with drug at a preset initial pressure and/or may be connected to a pressure driven displacement pump or a gas generating electrolytic cell that may continuously apply pressure to reservoir 310 after it has been implanted in the patient. Other membranes may permeate a drug on principles of osmosis. Further, reservoirs 310 may include iontophoresis membranes which have a permeability controlled by an applied electric field.

As shown in FIG. 3, reservoirs 310 may comprise rounded cavities along the length of electrodes 212. Reservoirs 312 may, however, have any shape and will likely be dictated based on the geometries of electrode 212. Further, while FIG. 3 shows each electrode 212 as having three reservoirs 310, any number of reservoirs may be included on an electrode. Moreover, not all electrodes 212 need have the same number of reservoirs 310 or even any reservoirs.

Even though electrodes 212 may include a reservoir, each electrode may still be able to detect neural signals and transfer those neural signals to processing unit 214 via wiring 216. To prevent reservoirs 310 from interfering with or affecting the detected neural signal, reservoirs 310 may be electrically insulated from the rest of electrode 212. In other embodiments, to prevent any type of interference, electrode array 210 may include electrodes dedicated to drug delivery (e.g., with reservoirs 310) and electrodes dedicated to detecting neural signals (e.g., with no reservoirs 310).

Further, reservoirs 310 may be filled during a manufacturing stage of electrode array 210 or may be filled or refilled during a surgical procedure. When array 210 includes multiple reservoirs 310, different drugs may be used. For instance, reservoirs 310-a, 310-b, and 310-c shown in FIG. 3 may each contain a different type of agent, such as a drug.

FIG. 4 illustrates a second exemplary embodiment of electrode array 210 having reservoirs for delivering agents or drugs. As shown in FIG. 4, electrode array 210 may include reservoirs 410 located at the base of each electrode 212. A respective delivery channel 420 connects each reservoir 410 to an opening 430 at the tip of the respective electrode 212. Thus, an agent or drug contained in reservoir 410 may be delivered to the patient through an electrode via its corresponding delivery channel 420. Reservoirs 410 may control the delivery of drug based on the pressure within reservoir 410, as described above with respect to FIG. 3. Further, like the embodiment of FIG. 3, reservoirs 410 may be filled during a manufacturing stage of electrode array 210 or may be filled or refilled during a surgical procedure.

For the embodiments of FIGS. 3 and 4, the arrangement of the different electrodes dedicated to the different types of uses may be determined according to the application at hand, but may include any of the following types of arrangements: (a) reservoir electrodes arranged around each detecting electrode; (b) detecting electrodes arranged around each reservoir electrode; and (c) reservoir electrodes arranged together in one portion of array 210 and detecting electrodes arranged together in another portion of array 210. Further, electrodes having reservoirs 310 or 410 may be configured to be separable from those electrodes without reservoirs, so that the reservoir type electrodes may be more easily replaced or refilled.

Another exemplary embodiment of an electrode array may include an array of sensors combined with cell capsules that penetrate or are otherwise placed in the brain or other body tissue. Encapsulated cells may treat a variety of disorders, such as Parkinson's disease. A cell capsule may include live cells in a sealed capsule. The cells may be capable of secreting a drug or other agent. The sealed capsule is configured to let nutrients/oxygen in and drugs and metabolites out. The cells remain immune privileged because of the capsule. One or more sealed capsules of live cells may be placed in the brain with, for example, the electrode array. The sensors of the array may detect and transmit information relating to the release of the agents and/or may regulate agent release through, for example, feedback control.

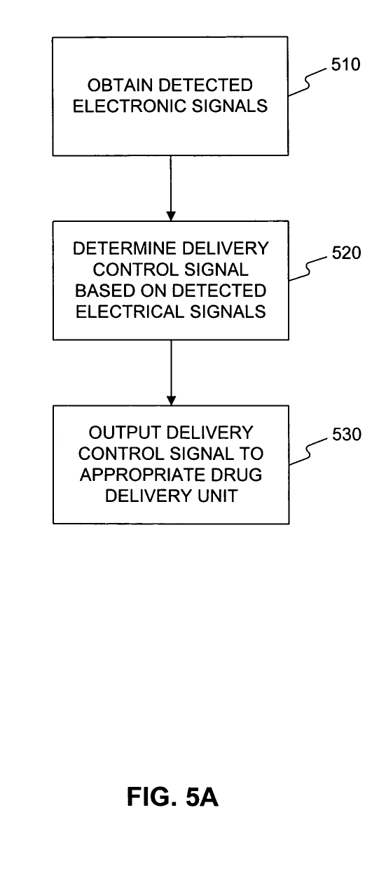

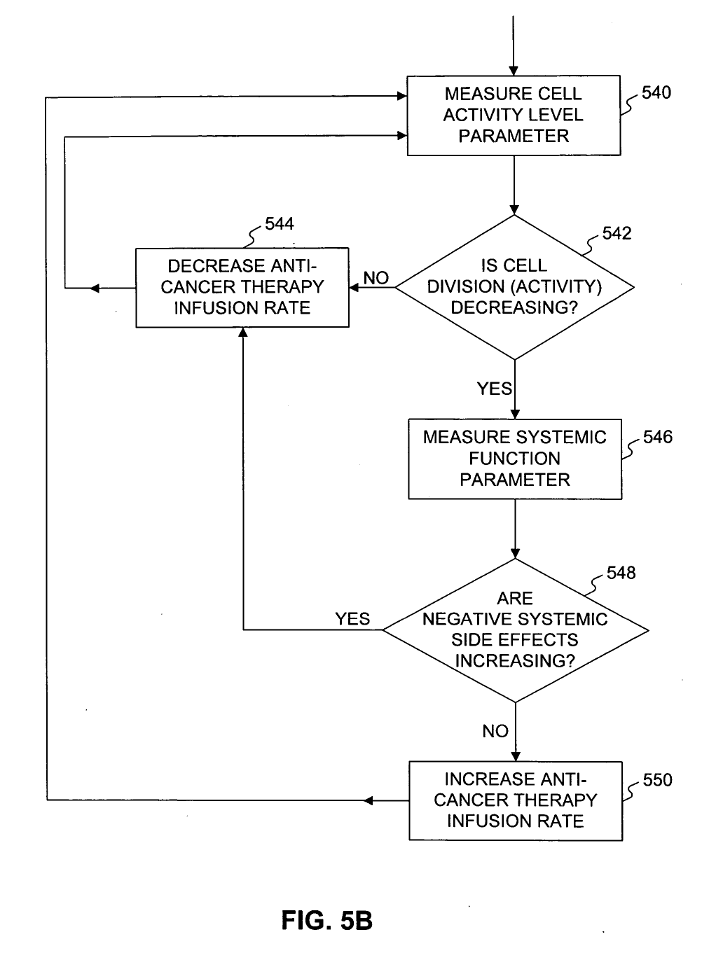

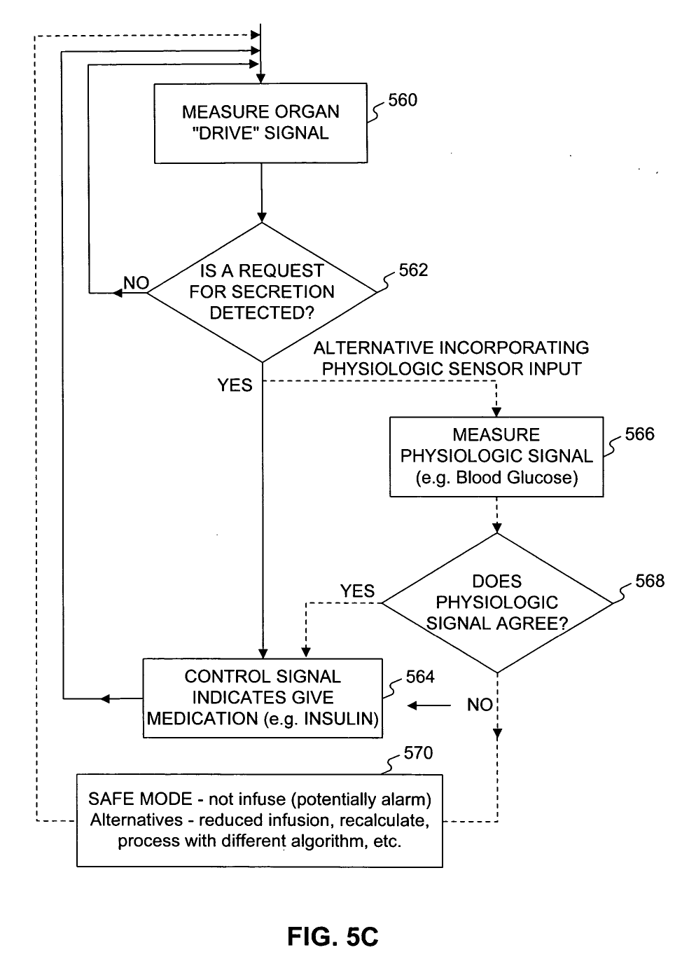

FIG. 5A illustrates a flow diagram of a method, consistent with the present invention, for delivering an agent to a patient through an implanted device. As shown in FIG. 5A, an implant 120, such as electrode array 210, obtains detected electrical signals from a patient (step 510). The detected signals may, for example, be neural signals obtained from an implant implanted in the patient's brain. As noted above with respect to FIG. 1, however, implants 120 may be located throughout the body and may detect electrical signals generated by other organs or tissue.

Processing unit 214 may then determine a delivery control signal based on the detected electrical signals (step 520). The delivery control signal may be used to control delivery of a biologic, therapeutic, or other agent. To determine the control signal, processing unit 214 may, for example, compare the detected neural signals to a drug delivery template for the patient. For instance, as noted above, processing unit 214 may refer to a look-up table having trigger values defining an amount and type of drug that should be delivered based upon a detected electrical signal. In other words, as noted above with respect to FIG. 2, the stored table values may be used to convert a detected biological electrical signal to an output control signal used for controlling drug delivery.

In systems consistent with the invention, processing unit 214 may generate a control signal that may provide continuous or semi-continuous delivery of the agent, including a control signal that is a two-state signal (e.g., on/off). The control signal also can be on-demand by the patient, allowing for voluntary control by the patient. Examples where this may be suitable include pain or sleep control therapies. Another example includes therapies relating to memory loss, where a memory enhancing drug may be delivered through a patient-induced action. Alternatively, the control signal can automatically control an agent delivery unit or semi-automatically control the delivery unit. Semi-automatic control may be combined with a patient or clinician confirmation step. In addition, the control signal may include a combination of voluntary patient control and automatic control.

The delivery control signal may include information controlling one or more of the following: a selected type of agent delivered, an amount of agent delivered, and a particular implant 120 to delivery the agent. For instance, as noted above, to control the delivery amount, the delivery control signal may reflect a desired delivery rate of the agent. The control signal may thus define an on/off state of agent delivery, whether the delivery is continuous or semi-continuous, a time schedule for agent delivery, and/or a concentration of the agent delivered.

Processing unit 214 may then output the delivery control signal to an appropriate drug delivery unit to control delivery of a drug (step 530). For example, the drug delivery unit may receive a control signal that includes information and instructions relating to agent delivery. The delivery unit then delivers the agent in a fashion instructed by the control signal. The delivery unit may be any suitable device capable of delivering an agent to a body, such as those shown in FIGS. 3 and 4. The delivery unit may be external to the patient and connected to the patient through a device like a catheter, may be internal to the patient (e.g., as shown in FIGS. 3 and 4), or a combination of external and internal devices. The delivery unit may be included within another component of the system, such as the signal sensor or detector, signal processor, or power supply.

The delivery unit may include a physician or other clinician who receives the control signal through a computer or other external means for receiving a signal from the patient (e.g., external processing unit 150), and then aids in the delivery of agent to the patient. For instance, as an alternative to or in conjunction with outputting the delivery control signal to an actual drug delivery unit, processing unit 214 may output drug delivery data to an external communication device. For instance, processing unit 214 may output the drug delivery data to a visual display (e.g., external processing unit 150 of FIG. 1) or to a printer (not shown) so that it may be viewed by a physician. From the displayed data, the physician may then determine a dosage of the drug to be given to the patient. Further, delivery units that are external to the patient may receive the control signal through a wired or wireless connection, through telemetry, or any other suitable method for communicating an electrical signal.

The agent to be delivered may be any biologic, therapeutic, or other agent, or any combination thereof. Such agents can include a drug, for example. The drugs may be used to treat any of a variety of conditions, diseases or disorders, including for example, neurological disorders, neuropsychiatric disorders including depression, diabetes, epilepsy, cancer, Parkinson's disease, Alzheimer's disease, ALS, cardiovascular disease, incontinence, obesity, eating disorders such as anorexia nervosa and bulimia, and others. Exemplary drugs suitable for use in systems and methods according to embodiments of the invention include various pain relievers, insulin, analgesics, antibiotics, chemotherapeutics, brain function and protection drugs, anti-depressant and other psychiatric mediations, anti-inflammatories, anti-convulsants, anxiolytics, anti-migraine drugs, anti-dementia drugs, drugs to treat vertigo, stimulants, cardiovascular medications, beta blockers, beta agonists, and neurotropic factors (such as glial-derived neurotropic factor GDNF, brain-derived neurotropic factor BDNF, ciliary neurotropic factor CNTF). Exemplary specific drugs include, but are not limited to, aspirin, cycloxygenase inhibitors, morphine, ketamine, fluoxetine, Zoloft, welbutrin, caffeine, Adderall, Dexedrine, Ritalin, modafininl, sutruamine, and guanfacine. Other drugs to treat any biological or other disorder of a patient may be used in connection with embodiments of the invention. Other such agents or drugs that may be used include biologic agents such as platelets, stem cells, bio-engineered vectors such as viruses, protein fragments such as immunoglobulins, vaccines, lipids, sugars, electrolytes, water or saline, and contrast agents.

FIG. 5B illustrates a flow diagram of an exemplary method, consistent with the present invention, for delivering an agent to a patient as part of a treatment. As described below, the method of FIG. 5B may be used to control the delivery of drug based on a detection of treated cell activity and systemic side effects on the patient. For example, the method of FIG. 5B may be used to control a chemotherapy treatment for cancerous tumors.

For instance, as shown in FIG. 5B, the method may detect a cell activity level parameter (step 540). The cell activity level parameter may be detected by a sensor located near cells in the patient that are being treated. The sensor may, for example, be a sensor similar to those described above with respect to FIGS. 2 to 4. Further, the cells being treated may any type of cells that a treatment is controlling the growth of, such as cancer cells or tumor cells. The sensor may thus be implanted in or near the region of interest in the patient (e.g., at the tumor site). The sensor (e.g., via its electrodes) would thus sense the electrical signals generated by the cell growth or division. Processor 214, for example, may then quantify the sensed electrical signals to generate the cell activity level parameter.

The cell activity parameter may then be compared to an activity threshold value to determine an effectiveness of the drug treatment therapy (step 542). For instance, the threshold comparison may determine whether cell activity is decreasing over time (i.e., the tumor is shrinking). In such a case, the cell activity parameter may be compared to a previously generated cell activity parameter. Alternatively, the cell activity parameter may be compared to a preset threshold value to determine if the generated parameter is within a desired range. Persons skilled in the art will appreciate, however, that the processing of step 542 may use other ways of determining an effectiveness of the drug treatment therapy.

If the processing of step 542 determines that the drug treatment therapy is not being effective, then the amount of delivered drug may be reduced (step 544). The drug may be delivered using a drug delivery unit such as those described above with respect to FIGS. 3 and 4. Further, the control of the drug delivered based on the comparison of step 542 may be implemented by using the processes described above with respect to steps 520 and 530 of FIG. 5A.

If, however, the processing of step 542 determines that the drug treatment is being effective, then a systemic function parameter may be detected (step 546). The systemic function parameter reflects a systemic function of the patient that may be affected by the drug delivery treatment. For example, a patient's neurological response, respiratory system, or cardiovascular system may be affected by a drug treatment. An additional sensor may thus be used to detect a biological signal associated with such a systemic function. For instance, a brain implant or EEG sensor could be used to detect a patient's neurological response. Other known sensors could be used to detect a measure of the patient's respiratory or cardiovascular system (e.g., a heart rate monitor). Based on the detected output from such a sensor, processor 214, for example, may then quantify the sensed biological signal to generate the systemic function parameter.

The systemic function parameter may then be compared to a systemic threshold value to determine whether systemic side-effects of the drug treatment therapy are increasing (step 548). For instance, the threshold comparison may determine whether cell activity is increasing over time (i.e., the heart rate is increasing) by comparing the parameter to a previously generated parameter. Alternatively, the systemic function parameter may be compared to a preset threshold value to determine if the generated parameter is within a desired range (e.g., that the heart rate is within a prescribed tolerance or that the patient is not having difficulty breathing). Persons skilled in the art will appreciate, however, that the processing of step 548 may use other ways of determining whether systemic side-effects are increasing.

If the drug treatment therapy is causing side-effects to increase, then processing proceeds to step 544, where the amount of delivered drug may be reduced. If, however, the drug treatment therapy is not causing side-effects to increase, then the amount of delivered drug may be increased (step 550), since it has already been determined (e.g., in step 542) that the therapy is being effective.

Further, the method of FIG. 5B may be used to identify optimal combinations of drugs and steroids that balance effectiveness of the drug treatment with low systemic side-effects. In doing so, other factors, such as the time of day to infuse a drug or agent, may be monitored to determine an optimal drug therapy.

FIG. 5C illustrates a flow diagram of an exemplary method, consistent with the present invention, for delivering an agent to a patient based on a detected neurological signal sent an organ of the patient. As shown in FIG. 5C, an organ “drive” signal may be detected (step 560). For example, a neurological sensor implanted in the patient's brain may be used to detect a neurological signal sent to an organ that secretes a biological agent. The sensor may also be located at or near, for example, the organ, or a body structure between the brain and organ, such as a nerve, that is communicating the detected neurological signal. If the organ drive signal then includes a request to secrete the biological agent (step 562), then processing proceeds to step 564 where, for example, processor 214 determines whether to output a delivery control signal for controlling a delivery unit to delivery the same biological unit. The processing of step 564 may be implemented using the processes described above with respect to steps 520 and 530 of FIG. 5A. In this way, systems consistent with the invention may be used to substitute a diseased or damaged organ (i.e., that does not adequately secrete the biological agent) with a drug delivery system for delivering the biological agent. As described above, an aspect of the invention thus uses the actual neurological signal generated to control the organ's secretion to also control the drug delivery unit.

An example of an organ in which the process of FIG. 5C may be used include controlling the secretion of insulin normally secreted by the pancreas. In such an embodiment, exemplary locations for a sensor may include cranial nerve nuclei, the vagus nerve, branches of the vagus nerve that are connected to the pancreas, autonomic ganglia that send nerves to the pancreas including sympathetic ganglia, celiac ganglion and potentially the superior mesanteric ganglia.

The method of FIG. 5C does not necessarily need to be used with a diseased or damaged organ. For instance, the process of FIG. 5C may be used to supplement the function of a healthy organ.

The method of FIG. 5C may also be implemented with an additional sensor measuring a physiologic signal (step 566). The physiological signal reflects an additional physiological measurement associated with the controlled secretion of the biological agent. In other words, the physiological signal is another measure of whether to secrete the biological agent. For example, for controlling the secretion of insulin, the physiological signal may be an output from a blood glucose sensor. The sensor may be placed in the blood stream or other location in the presence of interstitial fluid, such as subcutaneous tissue. Depending upon the biological agent delivered, however, the physiological signal may be output from other types of sensors, such as a respiratory sensor, an EKG sensor, or blood analysis sensor. Based on the detected output from such a sensor, processor 214, for example, may then quantify the sensed biological signal to generate the systemic function parameter.

At step 568, the system may then determine whether the physiological signal agrees with the determination made at step 564 on whether to output a delivery control signal. If the physiological signal agrees (e.g., it indicates that the biological agent should be delivered, when step 564 outputs a delivery signal), then the biological agent is delivered to the patient. If, however, the physiological signal is not in agreement, then a safe mode is entered where the biological agent is not delivered to the patient (step 570). The safe mode may comprise outputting an alarm to the patient, reducing the agent delivery amount, and/or to recalculate whether to deliver the biological agent at a later time or by using different decision criteria implemented by step 564.

A further exemplary embodiment of the method of FIG. 5C may include detecting electrical signals originating from the brain and being sent to a gland, such as a pituitary gland, commanding the gland to secrete a biologic substance, such as a cortisone or steroid-like substance. This may be especially suitable for patients with Addison's disease or other gland disorder in which the gland will not secrete the requested agent. As indicated above with respect to FIG. 5C, the electrical signal may be intercepted by a sensor at or near, for example, the brain, the gland, or a body structure therebetween, such as a nerve, that is communicating that signal. The sensed electrical signal may be processed to a signal that controls delivery of the requested agent from a drug delivery apparatus, such as a drug pump.

An additional embodiment of the invention includes systems, apparatuses, and related methods that detect neural or other electrical signals generated within a patient's body, and analyze and/or process those signals to generate a first signal that controls a controlled device and a second signal that controls delivery of a biologic, therapeutic, or other agent, such as a drug. The second signal may be used to deliver a drug that, for example, will affect the performance or other parameter of the controlled device.

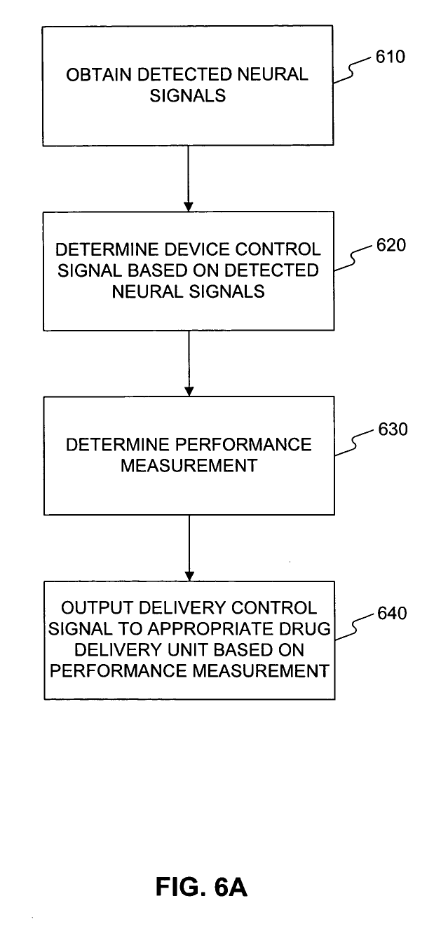

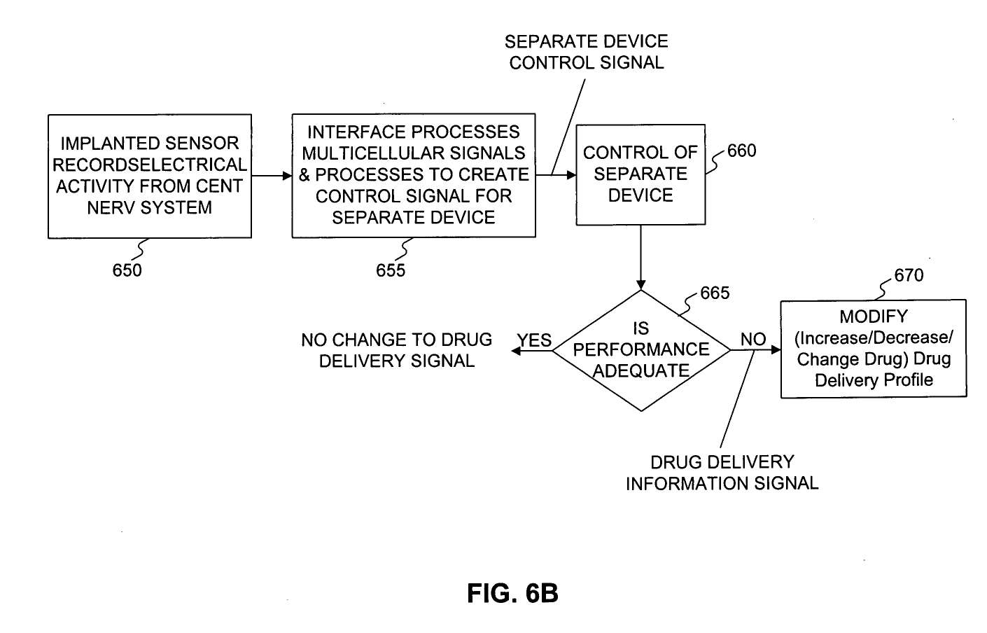

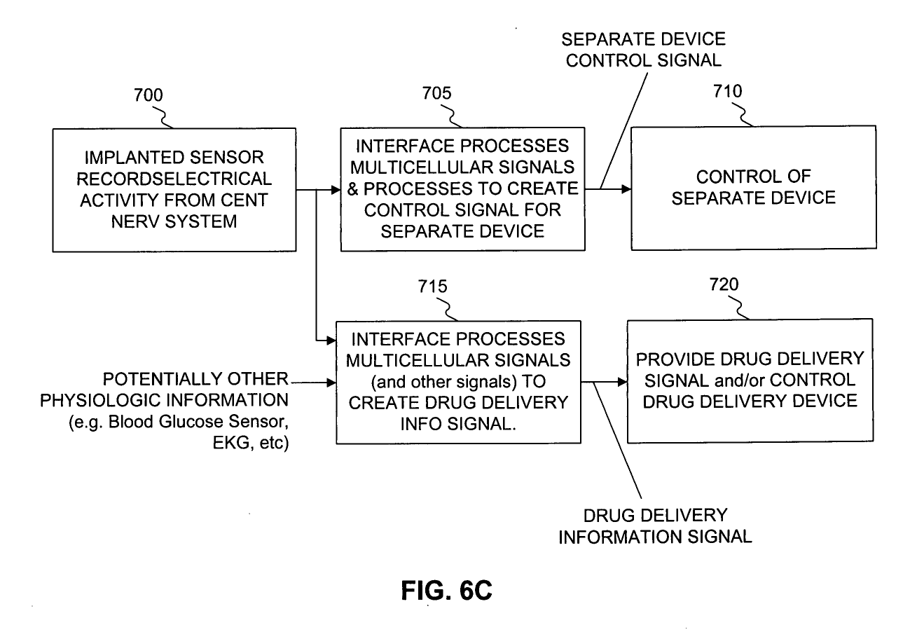

FIG. 6A illustrates a flow diagram of a method, consistent with the present invention, for delivering an agent to a patient to improve performance of a brain implant device. As shown in FIG. 6A, an implant 120, such as electrode array 210, obtains detected neural signals from a patient (step 610). The detected signals may, for example, be neural signals obtained from an implant implanted in the patient's brain.

From the detected neural signals, processing unit 214 may determine a device control signal for controlling a controlled device (step 620). For example, the controlled device may be any device that can be controlled by a processed electrical signal, including prosthetic limbs, stimulators for the muscles, organs, heart, or other part of the body, cardiac pacing devices, transcutaneous electrical nerve stimulators (TENS) for controlling pain, magnetic therapy devices, radiation delivery devices, a computer or other externally controlled apparatus, or a robotic device, computer control devices (e.g., keyboards, mice, etc.), communication devices, or transportation devices (e.g., automobiles, wheelchairs, etc.). In such a case, implant 120 may be a brain implant such as electrode array 210 having electrodes 212 extending into brain 220 to detect the electrical neural signals generated from the neurons located in proximity to the electrode's placement within the brain. Neurons may generate such signals when, for example, the brain instructs a particular limb to move in a particular way. Above U.S. Pat. No. 6,171,239 to Humphrey discloses methods for determining a device control signal based on detected neural signals, the entire disclosure of which is incorporated by reference herein.

Processing unit 214 then determines a parameter, such as a performance measurement, associated with either the detected neural signals or the control signal, determined as part of step 620, for controlling the controlled device (step 630). For example, the system may analyze the performance of a medical device controlled by the device control signal and/or the device control signal itself, or the input to or derivatives of the control signal, to generate (as part of step 640 below) the delivery control signal for controlling delivery of a drug.

To determine the performance of the brain implant, processing unit 214 may compare the controlled device's control signal with a performance template. The performance template may include criteria defining a target performance of the controlled device's control signal (e.g., whether the control signal is erratic). The performance criteria may also be dependent upon the operation mode of the brain implant. For example, the performance template may include respective set of performance criteria depending upon whether the brain implant is in a calibration mode, a training mode, or an operation mode. Criteria for determining performance include those described below with respect to FIG. 6B. If the control signal for the device does not meet the performance criteria included in the performance template, then processing unit 214 may output a delivery control signal causing an agent or drug to be delivered to the patient.

Processing unit 214 may determine a performance measurement by monitoring a parameter of the controlled device. For example, a performance template may include criteria defining a target performance of the controlled device itself. As above, if the controlled device does not meet the performance criteria included in the performance template, then processing unit 214 may output a delivery control signal causing an agent or drug to be delivered to the patient.

Accordingly, processing unit 214 then determines a delivery control signal based on the determined performance measurement (step 640). For instance, a particular drug or combination of drugs may be used to treat the patient to improve performance of an implant 120, as described in more detail with respect to FIG. 6B.

Thus, based on the determine performance measurement, processing unit 214 determines what drug may improve performance of the medical device or other controlled device and outputs a delivery control signal to the appropriate delivery unit for dispensing the appropriate type, amount, rate, etc. of the drug to the patient. Processing then reverts back to step 630, where another performance measurement is made.

FIGS. 6B and 6C further illustrate systems consistent with the invention as discussed above with respect to FIG. 6A.

FIG. 6B is a flowchart of an additional embodiment of the process steps implemented by a brain machine interface system that produces a drug delivery signal to improve control of a device. In a first step 650, an implanted sensor records electrical activity from the central nervous system or other source of electrical information within the body. For example, the sensor may be implanted proximate the brain to receive multicellular signals from the motor cortex. Those signals may be conveyed to a processor (e.g., processor 214) that processes the signals to create a control signal for a separate device, as depicted in process step 655. The processor may include any suitable processing steps as described throughout this disclosure, including at least signal selection, filtering, amplification, and mathematical processing. The control signal then is transmitted to a separate device. The separate device may be any device that may be controlled by processed electrical signals from a body, including at least the various devices mentioned throughout this disclosure. In step 660, the control signal controls the operation of the device.

In step 665, the system then may analyze either or both of the control signal and performance of the device to determine whether a drug delivery profile should be altered in a way that will alter performance of the system, including at least control and/or performance of the device. For example, the system may include suitable components to analyze one or more control signal properties, including its stability, continuity, amount of rejected data, or other indicators of the control signal. More specifically, the system may analyze whether the filtering of the signals is discarding a higher amount of data that is not useful, whether the spike activity of the signal is increasing or decreasing, and/or whether noise of the control signal is increasing or decreasing. The system may analyze whether one or more of these control signal properties is changing to unsatisfactory levels that may affect performance of the system and specifically control or performance of the device.

The system also may analyze performance of the device itself. For example, the system may analyze whether the device is meeting performance standards. Those standards may be stored within a processor or other component of the system in a table or other format for easy access and comparison to measured performance. As a specific example, a patient, through the brain machine interface system, may control typing within a computer at a satisfactory level of 100 words per minute for a sustained period and then control typing at 40 words per minute. The system may store a threshold value above 40 words per minute (and below 100 words per minute) that would indicate that the drug delivery profile to the patient should be altered to improve the performance of the patient's control of the typing. Similarly, the system may analyze whether the device is showing trends in its performance, such as a sharp decrease in the amount of words per minute that a patient is able to type through the system. As a further example, the system may be used to analyze whether performance of a controlled wheelchair is meeting performance standards by measuring whether the wheelchair is bumping into objects, has erratic speed control, or is exhibiting other behaviors that would indicate unsatisfactory performance.

Based on the system's analysis of either or both of the control signal and performance of the device, the system will determine whether performance of the system is adequate (step 665) and, if not, create and send a drug delivery information signal to a drug delivery device to modify the drug delivery profile (step 670). The signal may initiate an increase or decrease in the amount or rate of delivery of a drug, a change in the drug being administered, or any other modification in the delivery profile described throughout this disclosure. For example, if the patient is controlling the typing of a number of words per minute below a set threshold, the drug delivery information signal may initiate the administration of a stimulant. In other example, sedatives, anti-depressants, or any of the other drugs or agents described in this disclosure may be administered, depending on the circumstance. In addition or as an alternative, the drug delivery information signal may be sent to a clinician in a suitable form for the clinician to decide on the drug delivery profile.

FIG. 6C is a flowchart of another embodiment of the process steps implemented by a brain machine interface system that produces a drug delivery signal to improve control of a device. In step 700, like step 650 in FIG. 6B, an implanted sensor records electrical activity from the central nervous system or other source of electrical information within the body. Those signals may be conveyed to a processor that processes the signals to create a control signal for a separate device, as depicted in process step 705 (like step 655 of FIG. 6B). The control signal then is transmitted to a separate device to control operation of the device, as depicted in step 710 (like step 660 in FIG. 6B).

Unlike the embodiment shown in FIG. 6B, the sensed electrical activity from the body may be conveyed to another processor that processes those signals to create a drug delivery information signal, as depicted in step 715. That processor may also receive other signals or input to create the drug delivery information signal. For example, as shown in FIG. 6C, other physiologic information, such as blood glucose levels obtained from a glucose sensor and/or EKG information, may be input to the processor. Or, additional multicellular signals may be input to this second processor. The processor then processes the sensed electrical signals, with or without any physiologic information input, to create a drug delivery information signal. For example, the processor may analyze the electrical signals to determine whether there have been changes in spike rate, spike amplitude, signal modulation, signal number, and/or other signal characteristics, and analyze the physiologic input for changes as well. The processor may determine whether the changes show trends or sharp increases or decreases, or are above or below preset thresholds.

Based on this analysis, the processor will create and send a drug delivery information signal to a drug delivery device to modify the drug delivery profile (step 720). As in step 670 of FIG. 6B, the signal may initiate an increase or decrease in the amount or rate of delivery of a drug, a change in the drug being administered, or any other modification in the delivery profile described throughout this disclosure.

An exemplary embodiment of processes of FIGS. 6A to 6C includes a system in which the controlled device includes an external computer. The system includes a sensor to detect electrical signals of a patient that may, for example, be unable to operate a manual, standard computer keyboard through conventional means (e.g., using fingers to press buttons). The sensed electrical signals may be neural signals containing information regarding intended keys to depress on a keyboard so as to perform computer functions, such as word processing. The sensed signals may be processed to generate a control signal that is transmitted, for example wirelessly, to a computer to perform the computer function. The system may further include structure for monitoring a parameter of the computer, including a performance characteristic such as the number of words per minute that the user is typing. That structure may be circuitry internal or external to the computer. The parameter information obtained from that monitoring may be transmitted to a processor and processed to a second control signal that, in turn, is transmitted to an agent delivery device. The agent delivery device may then deliver a drug that will enhance or otherwise alter the parameter. For example, a substantial decrease in the number of words per minute may indicate that the user has tired. The agent delivery device may select and deliver a stimulant, such as caffeine, to improve the performance of the system.

The following examples of embodiments of the invention are non-limiting. The invention encompasses numerous other applications of delivery of an agent to a patient based on detecting a patient's electrical signals and processing those signals to obtain a control signal for agent delivery.

An exemplary embodiment of the present invention includes performing hypothalamic recording to treat obesity. A sensor may be used to detect a signal for satiety and the signal may be processed to a signal that controls delivery of a drug that controls hunger. To obtain the electrical signals that would have the needed information, a sensor may be placed at or near lateral nuclei of hypothallumus, ventromedial nuclei of the hypothallamus, dorsal motor nucleus of vagus in the brain stem, solitary nucleus in the brain stem, cranial nerve nuclei of the brain stem, nucleus ambiguus, nerves that innervate the pancreas, the gallbladder, or the gastrointestinal organs. Over delivery of the drug may be avoided based on the satiety signal.

Another exemplary embodiment of the present invention includes sensing electrical signals that indicate the onset of an undesired state of a psychiatric disorder. The sensed signal may be processed to a signal that controls intrathecal release of a drug, such as glial-derived neurotropic factor GDNF, brain-derived neurotrohpic factor BDNF, or ciliary neurotropic factor CNTF, either in a continuous and/or bolus fashion. As with other embodiments, the agent delivery may be patient activated or automatically activated by the control signal.

The invention encompasses numerous other applications of delivery of an agent to a patient that will affect the performance of a controlled device. The following examples of embodiments of the invention are non-limiting.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for treating a body, comprising:
detecting electrical signals from a first part of the body;
processing the detected electrical signals to generate a first control signal;
controlling a device based on the first control signal;
generating a second control signal; and
providing information relating to delivery of an agent to the body, wherein the information is based on the second control signal.

2. The method of claim 1, wherein detecting electrical signals includes detecting electrical signals generated by brain neural signals.

3. The method of claim 1, wherein detecting electrical signals includes detecting electrical signals generated by voluntary control.

4. The method of claim 1, wherein detecting electrical signals includes detecting electrical signals generated by nerves.

5. The method of claim 1, wherein detecting electrical signals includes detecting electrical signals generated by a tumor.

6. The method of claim 1, wherein detecting electrical signals includes detecting electroencephalogram (EEG) signals.

7. The method of claim 1, wherein detecting electrical signals includes detecting neuron spike signals.

8. The method of claim 1, wherein detecting electrical signals includes detecting local field potentials.

9. The method of claim 1, wherein detecting electrical signals includes detecting electrocortigram (EcoG) signals.

10. The method of claim 1, wherein the device includes a computer.

11. The method of claim 1, wherein the device includes a prosthetic limb.

12. The method of claim 1, wherein the device includes a body part.

13. The method of claim 1, wherein the second control signal is used to display information for delivering the agent.

14. The method of claim 1, wherein the second control signal is based on a signal transmitted from the device.

15. The method of claim 1, wherein the second control signal is based on data relating to the device.

16. The method of claim 1, further comprising monitoring a parameter of the device.

17. The method of claim 16, wherein the second control signal is generated based on the monitored parameter.

18. The method of claim 17, wherein the monitored parameter reflects a performance value of the control over the device.

19. The method of claim 17, further comprising delivering the agent to the body to alter the monitored parameter of the device.

20. The method of claim 19, wherein the monitored parameter is a performance value of the device, and delivering the agent improves the performance value of the device.

21. The method of claim 1, further comprising continuously monitoring a parameter of the device and continuously generating second control signals based on the continuous monitoring of the parameter.

22. The method of claim 1, wherein the agent is a drug.

23. The method of claim 1, wherein the second control signal includes information relating to a type of the agent delivered.

24. The method of claim 1, wherein the second control signal includes information relating to a selection of the agent from a group of agents.

25. The method of claim 1, wherein the second control signal includes information relating to an amount of the agent to deliver.

26. The method of claim 1, wherein the second control signal includes information relating to a rate of delivery of the agent.

27. The method of claim 1, wherein the second control signal includes information relating to an on/off state of delivery of the agent.

28. The method of claim 1, further comprising implanting a sensor in the body proximate the part of the body, the sensor for detecting the electrical signals.

29. The method of claim 28, wherein the sensor includes an array of electrodes.

30. The method of claim 29, wherein the sensor includes a delivery unit to deliver the agent.

31. The method of claim 30, wherein the delivery unit includes a reservoir associated with at least one electrode.

32. The method of claim 31, wherein the reservoir is configured to store the agent.

33. The method of claim 31, wherein the second control signal controls the delivery of the agent to the body from the reservoir.

34. The method of claim 31, wherein the reservoir includes a membrane through which the agent may permeate.

35. The method of claim 1, further comprising implanting a delivery unit in the body proximate to a second body part to which the agent is delivered.

36. The method of claim 30, further comprising implanting a processor in the body and connecting the processor to the sensor and the delivery unit, wherein the processor is configured to generate the first and second control signals.

37. The method of claim 36, wherein the sensor includes the processor.

38. The method of claim 36, wherein the delivery unit includes the processor.

39. The method of claim 36, wherein the processor includes a first processor associated with the sensor to generate the first control signal, and a second processor associated with the delivery unit to generate the second control signal.

40. The method of claim 1, wherein the information is provided to a practioner.

41. The method of claim 1, wherein the first part of the body is a brain.

42. The method of claim 1, wherein the first part of the body is a portion of a central nervous system.

43. The method of claim 1, wherein the first part of the body is a body organ.

44. The method of claim 1, wherein the first part of the body is bone marrow.

45. The method of claim 1, wherein generating the second control signal includes accessing a table of values stored in a processor.

46. The method of claim 45, wherein the table of values includes values that control delivery of the agent.

47. The method of claim 45, wherein the values are used to convert the detected electrical signals to the second control signal.

48. The method of claim 45, further comprising changing the values in the table.

49. The method of claim 48, wherein the values in the table are changed based on a measured parameter of the device.

50. A system for treating a body, comprising:
a sensor configured to be proximate to a first part of the body generating electrical signals and to detect the electrical signals;
a first processor connected to the sensor for processing the detected electrical signals to generate a first control signal;
a device configured to receive the first control signal and be controlled by the first control signal; and
a second processor configured to generate a second control signal based on a monitored parameter of the device and to provide information relating to delivery of an agent to the body based on the second control signal.

51. The system of claim 50, wherein the sensor detects electrical signals generated by brain neural signals.

52. The system of claim 50, wherein the sensor detects electrical signals generated by voluntary control.

53. The system of claim 50, wherein the sensor detects electrical signals generated by nerves.

54. The system of claim 50, wherein the sensor detects electrical signals generated by a tumor.

55. The system of claim 50, wherein the sensor detects electroencephalogram (EEG) signals.

56. The system of claim 50, wherein the sensor detects neuron spike signals.

57. The system of claim 50, wherein the sensor detects local field potentials.

58. The system of claim 50, wherein the sensor detects electrocortigram (EcoG) signals.

59. The system of claim 50, wherein the device includes a computer.

60. The system of claim 50, wherein the device includes a prosthetic limb.

61. The system of claim 50, wherein the device includes a body part.

62. The system of claim 50, wherein the first processor includes the second processor.

63. The system of claim 50, wherein the second processor is connected to the device.

64. The system of claim 50, wherein the second processor is configured to receive information relating to the monitored parameter.

65. The system of claim 50, wherein the second control signal is used to display information for delivery of the agent.

66. The system of claim 50, wherein the second control signal is based on a signal transmitted from the device.

67. The system of claim 50, wherein the second control signal is based on data relating to the device.

68. The system of claim 50, wherein the second processor monitors the parameter of the device.

69. The system of claim 50, further comprising an agent delivery unit configured to receive the second control signal.

70. The system of claim 50, wherein the monitored parameter reflects a performance value of the control over the device.

71. The system of claim 50, wherein the agent alters the monitored parameter of the device.

72. The system of claim 71, wherein the monitored parameter is a performance characteristic of the device, and the second control signal causes delivery of the agent to improve the performance characteristic of the device.

73. The system of claim 50, wherein the second processor continuously monitors the parameter of the device and continuously generates second control signals based on the continuous monitoring of the parameter.

74. The system of claim 50, wherein the agent is a drug.

75. The system of claim 50, wherein the second control signal includes information relating to a type of the agent delivered.

76. The system of claim 50, wherein the second control signal includes information relating to a selection of the agent from a group of agents.

77. The system of claim 50, wherein the second control signal includes information relating to an amount of the agent to deliver.

78. The system of claim 50, wherein the second control signal includes information relating to a rate of delivery of the agent.

79. The system of claim 50, wherein the second control signal includes information relating to an on/off state of delivery of the agent.

80. The system of claim 50, wherein the sensor includes an array of electrodes.

81. The system of claim 80, wherein the sensor includes an agent delivery unit.

82. The system of claim 81, wherein the agent delivery unit includes a reservoir associated with at least one electrode.

83. The system of claim 82, wherein the reservoir connects to the second processor to receive the second control signal.

84. The system of claim 50, wherein the second processor is configured to transmit information relating to the second control signal to a practicioner.

85. The system of claim 69, wherein the agent delivery unit includes a pump.

86. The system of claim 50, wherein the second processor communicates with an agent delivery unit through a wireless connection.

87. The system of claim 50, wherein the second processor communicates with an agent delivery unit through a wired connection.

88. The system of claim 50, wherein the first processor communicates with the device through a wireless connection.

89. The system of claim 50, wherein the first processor communicates with the device through a wired connection.

90. The system of claim 69, wherein the agent delivery unit includes the agent.

91. The system of claim 69, wherein the agent delivery unit includes a reservoir configured to store the agent.

92. The system of claim 91, wherein the second control signal controls the delivery of the agent to the body from the reservoir.

93. The system of claim 91, wherein the reservoir includes a membrane through which the agent may permeate.

94. The system of claim 69, wherein the agent delivery unit is configured to be implanted in the body proximate to where the agent is delivered.

95. The system of claim 50, wherein the sensor includes the first processor.

96. The system of claim 69, wherein the agent delivery unit includes the second processor.

97. The system of claim 50, wherein the sensor includes the first processor and the second processor.

98. The system of claim 50, wherein the first part of the body is a brain.

99. The system of claim 50, wherein the first part of the body is a tumor.

100. The system of claim 50, wherein the first part of the body is a portion of a central nervous system.

101. The system of claim 50, wherein the first part of the body is a body organ.

102. The system of claim 50, wherein the first part of the body is bone marrow.

103. The system of claim 50, wherein the second processor generates the second control signal using a stored table of values.

104. The system of claim 103, wherein the table of values includes values that control delivery of the agent.

105. The system of claim 103, wherein the values are used to convert the detected electrical signals to the second control signal.

106. The system of claim 103, wherein the second processor is configured to permit the values in the table to be changed.

107. The system of claim 106, wherein the values in the table may be changed based on the monitored parameter of the device.

108. The system of claim 69, wherein the agent delivery unit includes a display for displaying agent delivery information to a practitioner.

109. A method for treating a body, comprising:
detecting neurological signals transmitted to a first part of the body, wherein the detected neurological signals relate to secretion of a first agent within the body;
processing the detected neurological signals to generate a first delivery control signal; and
delivering a second agent to the body based on the first delivery control signal.

110. The method of claim 109, wherein the first and second agents are the same.

111. The method of claim 109, further including sensing a physiological signal of the body.

112. The method of claim 111, further including generating the first delivery control signal based on the sensed physiological signal and the detected neurological signals.

113. The method of claim 112, wherein the generating of the first delivery control signal includes using the sensed physiological signal to confirm a value of the first delivery control signal generated based on the detected neurological signals.

114. The method of claim 113, wherein the first delivery control signal is off if the physiological signal does not confirm the value of the first delivery control signal generated based on the sensed electrical signals.

115. A method for treating a body, comprising:
detecting electrical signals generated by a first part of the body;
processing the sensed electrical signals to generate a performance value reflecting a performance of an applied treatment to the body;
determining a delivery control signal based on the performance value; and
delivering an agent to the body based on the delivery control signal.

116. The method of claim 115, wherein the first part of the body is a tumor location.

117. The method of claim 116, wherein the performance value reflects whether the applied therapy is reducing the tumor.

118. The method of claim 115, wherein the performance value is compared to a threshold to determine the delivery control signal.

119. The method of claim 115, further including determining a system function parameter reflecting a systemic function of the body affected by the applied treatment.

120. The method of claim 119, wherein the systemic function reflects a neurological response of the body.

121. The method of claim 119, wherein the systemic function reflects a respiratory response of the body.

122. The method of claim 119, wherein the systemic function reflects a cardiovascular response of the body.

123. The method of claim 119, wherein the systemic function parameter reflects a side-effect of the applied treatment.

124. The method of claim 119, wherein determining the delivery control signal includes determining the delivery control signal based on the systemic function parameter.

125. The method of claim 124, wherein the delivery control signal causes delivery of the agent when the systemic function parameter reflects the applied treatment has resulted in acceptable side-effects.

126. A method for treating a body, comprising:
detecting electrical signals from a part of the body;
processing the detected electrical signals to generate a first control signal;
controlling a device based on the first control signal;
monitoring a parameter of the device;
comparing the parameter to a value;
generating a second control signal based on the comparison of the parameter to the value; and
providing information relating to delivery of an agent to the body, wherein the information is based on the second control signal.

127. The method of claim 126, wherein the parameter is a performance characteristic of the device.

128. The method of claim 127, wherein the value is a performance standard stored in a processor.

129. The method of claim 128, wherein the performance standard is a threshold, and comparing the parameter to the value includes determining whether the performance characteristic is above the threshold.

130. The method of claim 128, wherein the performance standard relates to a trend in the parameter.

131. The method of claim 126, further comprising delivering the agent to the body.

132. The method of claim 131, wherein delivering the agent alters the parameter.

133. A method for treating a body, comprising:
detecting electrical signals from a part of the body;
processing the detected electrical signals to generate a first control signal;
controlling a device based on the first control signal;
monitoring a parameter of at least one of the detected electrical signals and the first control signal;
comparing the parameter to a value;
generating a second control signal based on the comparison of the parameter to the value; and
providing information relating to delivery of an agent to the body, wherein the information is based on the second control signal.

134. The method of claim 133, wherein the parameter is stability of the first control signal.

135. The method of claim 133, wherein the parameter is a spike activity of the detected electrical signals.

136. The method of claim 133, wherein the parameter the amount of data filtered to generate the first control signal.

137. The method of claim 133, wherein the parameter is a noise level of the first control signal.

138. The method of claim 133, wherein the value is a threshold.

139. The method of claim 133, wherein the value relates to a trend in the parameter.

140. The method of claim 133, further comprising delivering the agent to the body.

141. The method of claim 140, wherein delivering the agent alters the parameter.

142. A system for treating a body, comprising:
a sensor configured to be proximate to a first part of the body generating electrical signals and to detect the electrical signals;
a first processor connected to the sensor for processing the detected electrical signals to generate a first control signal;
a device configured to receive the first control signal and be controlled by the first control signal; and
a second processor configured to generate a second control signal based on a measured parameter of at least one of the detected electrical signals and the first control signal, the second processor configured to provide information relating to delivery of an agent to the body based on the second control signal.

143. The system of claim 142, wherein the parameter is stability of the first control signal.

144. The system of claim 142, wherein the parameter is a spike activity of the detected electrical signals.

145. The system of claim 142, wherein the parameter is the amount of data filtered to generate the first control signal.

146. The system of claim 142, wherein the parameter is a noise level of the first control signal.

147. The system of claim 142, further comprising an agent delivery unit configured to receive the information relating to the second control signal.

148. The system of claim 147, wherein delivering the agent alters the parameter.

149. The system of claim 142, wherein the first processor includes the second processor.
